(19) United States
(12) Patent Application Publication
(10) Pub. No.: US 2023/0391865 A1
(43) Pub. Date: Dec. 7, 2023

(54) ANTI-CANCER INHIBITORY ANTIBODIES

(71) Applicant: THE REGENTS OF THE UNIVERSITY OF CALIFORNIA, Oakland, CA (US)

(72) Inventor: Tannishtha Reya, San Diego, CA (US)

(21) Appl. No.: 18/249,170

(22) PCT Filed: Oct. 14, 2021
(86) PCT No.: PCT/US2021/055055

§ 371 Date: Apr. 14, 2023

Related U.S. Application Data
(60) Provisional application No. 63/092,384, filed on Oct. 15, 2020.

(51) Int. Cl. C07K 16/28; A61P 35/02

(52) U.S. Cl. CPC C07K 16/28; A61P 35/02; A61K 2039/505

(57) ABSTRACT

Provided are inhibitory antibodies against Tetraspanin-3 (TSPAN3), compositions containing the same, and methods of using the same for the treatment of TSPAN3-dependent cancers, including leukemia and solid cancers.

### CROSS-REFERENCE TO RELATED APPLICATIONS

This application claims the benefit of U.S. Provisional Patent Application No. 63/092,384, filed on Oct. 15, 2020, the contents of which are incorporated by reference in their entirety.

### TECHNICAL FIELD

The present technology relates to the inhibition of cancer cell growth and treatment of cancer using inhibitory antibodies against Tetraspanin-3 (TSPAN3).

### BACKGROUND

Many types of cancer, including leukemia, are frequently resistant to current treatments and thus remain lethal. As a result, a need exists for identification and development of novel molecular targets and/or therapeutic agents for improved cancer treatment.

TSPAN3 is a member of the transmembrane 4 superfamily, also known as the tetraspanin family. Most of the proteins in this family are cell-surface proteins, characterized by the presence of four hydrophobic domains, and mediate signal transduction events that play a role in the regulation of cell development, activation, growth, and motility.

### SUMMARY

The present technology relates to antibodies that specifically bind to TSPAN3 and/or inhibit TSPAN3 function, as well as compositions and methods for using the same for the treatment of cancers that express TSPAN3, such as leukemia and solid cancers.

In some aspects, provided are antibodies that specifically bind to TSPAN3.

In some embodiments, the antibody comprises a heavy chain variable region comprising at least one complementarity-determining region (CDR) having an amino acid sequence selected from the group consisting of SEQ ID NOs: 3, 4, 5, 8, 9, 10, 13, 14, and 15.

In some embodiments, the antibody comprises a heavy chain variable region comprising at least two CDRs, each having an amino acid sequence selected from the group consisting of SEQ ID NOs: 3, 4, 5, 8, 9, 10, 13, 14, and 15.

In some embodiments, the antibody comprises a heavy chain variable region comprising at least three CDRs, each having an amino acid sequence selected from the group consisting of SEQ ID NOs: 3, 4, 5, 8, 9, 10, 13, 14, and 15.

In some embodiments, the antibody comprises a light chain variable region comprising at least one CDR having an amino acid sequence selected from the group consisting of SEQ ID NOs: 18, 19, 20, 23, 24, 25, 28, 29, 30, 33, 34, and 35.

In some embodiments, the antibody comprises a light chain variable region comprising at least two CDRs, each having an amino acid sequence selected from the group consisting of SEQ ID NOs: 18, 19, 20, 23, 24, 25, 28, 29, 30, 33, 34, and 35.

In some embodiments, the antibody comprises a light chain variable region comprising at least three CDRs, each having an amino acid sequence selected from the group consisting of SEQ ID NOs: 18, 19, 20, 23, 24, 25, 28, 29, 30, 33, 34, and 35.

In some embodiments, the antibody comprises an amino acid sequence that is at least 70% identical to an amino acid sequence selected from the group consisting of SEQ ID NOs: 2, 7, 12, 17, 22, 27, and 32.

In some embodiments, the antibody comprises a heavy chain variable region comprising an amino acid sequence that is at least 70% identical to an amino acid sequence set forth in SEQ ID NO: 2, 7, or 12.

In some embodiments, the antibody comprises a light chain variable region comprising an amino acid sequence that is at least 70% identical to an amino acid sequence set forth in SEQ ID NO: 17, 22, 27, or 32.

In some embodiments, the antibody comprises (a) a heavy chain comprising CDRs having amino acid sequences set forth in SEQ ID NOs: 3-5 and a light chain comprising CDRs having amino acid sequences set forth in SEQ ID NOs: 18-20; (b) a heavy chain comprising CDRs having amino acid sequences set forth in SEQ ID NOs: 8-10 and a light chain comprising CDRs having amino acid sequences set forth in SEQ ID NOs: 28-30; or (c) a heavy chain comprising CDRs having amino acid sequences set forth in SEQ ID NOs: 13-15 and a light chain comprising CDRs having amino acid sequences set forth in SEQ ID NOs: 33-35.

In some embodiments, the antibody comprises (a) a heavy chain variable region comprising an amino acid sequence that is at least 70% identical to an amino acid sequence set forth in SEQ ID NO: 2 and a light chain variable region comprising an amino acid sequence that is at least 70% identical to an amino acid sequence set forth in SEQ ID NO: 17; (b) a heavy chain variable region comprising an amino acid sequence that is at least 70% identical to an amino acid sequence set forth in SEQ ID NO: 7 and a light chain variable region comprising an amino acid sequence that is at least 70% identical to an amino acid sequence set forth in SEQ ID NO: 27; or (c) a heavy chain variable region comprising an amino acid sequence that is at least 70% identical to an amino acid sequence set forth in SEQ ID NO: 12 and a light chain variable region comprising an amino acid sequence that is at least 70% identical to an amino acid sequence set forth in SEQ ID NO: 32.

In some embodiments, the antibody is a monoclonal antibody.

In some embodiments, the antibody inhibits TSPAN3 function.

In some embodiments, the antibody is a humanized antibody.

In some aspects, provided are pharmaceutical compositions comprising an antibody according to various embodiments disclosed herein.

In some aspects, provided are nucleic acids comprising a nucleotide sequence that encodes an antibody according to various embodiments disclosed herein. In some embodiments, the nucleotide sequence is at least 70% identical to a nucleotide sequence selected from the group consisting of SEQ ID NOs: 1, 6, 11, 16, 21, 26, and 31. In some embodiments, the nucleic acid is inserted into a vector.

In some aspects, provided are host cells containing a nucleic acid according to various embodiments disclosed herein. In some embodiments, the host cell produces the antibody encoded by the nucleic acid.

In some aspects, provided are compositions comprising a host cell according to various embodiments disclosed herein.

In some aspects, provided are methods of treating and/or preventing a cancer that expresses TSPAN3 in a subject in need thereof, comprising administering to the subject a therapeutically effective amount of an antibody, a pharmaceutical composition, or a composition according to various embodiments disclosed herein.

In some embodiments, the cancer is a hematologic malignancy. In some embodiments, the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

In some embodiments, the cancer is a solid cancer. In some embodiments, the solid cancer is selected from the group consisting of pancreatic cancer, glioma, glioblastoma, colorectal cancer, thyroid cancer, stomach cancer, ovarian cancer, melanoma, endometrial cancer, lung cancer, renal cancer, cervical cancer, prostate cancer, breast cancer, urothelial cancer, testicular cancer, head and neck cancer, liver cancer, and esophageal cancer.

### BRIEF DESCRIPTION OF THE DRAWINGS

FIG. 1 shows a schematic of a strategy for generating inhibitory TSPAN3 antibodies according to an embodiment disclosed and described herein.

FIG. 2 shows a functional analysis of various hybridoma clones on primary patient samples according to an embodiment disclosed and described herein.

FIG. 3 shows the impact of purified TSPAN3 monoclonal antibodies (mAbs) on the growth of primary leukemia patient cells in vitro according to an embodiment disclosed and described herein.

FIG. 4 is a compilation of data showing that TSPAN3 mAbs inhibited growth of various primary patient myeloid leukemia samples according to an embodiment disclosed and described herein.

FIG. 5 shows the impact of TSPAN3 mAbs on the growth of normal human CD34+ hematopoietic cells according to an embodiment disclosed and described herein.

FIG. 6 shows the treatment of human leukemia cells in vivo by TSPAN3 mAbs according to an embodiment disclosed and described herein.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200 but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

### Definitions

Unless otherwise specified, each of the following terms has the meaning set forth in this section.

The conjunctions “or” and “and/or” are used interchangeably.

The term “about,” as used herein when referring to a measurable value, such as an amount or concentration and the like, is meant to encompass variations of 20%, 10%, 5%, 1% 0.5%, or even 0.1% of the specified amount.

The term “antibody” is used to denote, in addition to natural antibodies, genetically engineered or otherwise modified forms of immunoglobulins, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single-chain variable fragment antibody (scFv), disulfide-linked Fv, single-domain antibody (sdAb), antigen-binding fragment (Fab), Fab′, F(ab′)2 fragment, or diabody. An scFv antibody is derived from an antibody by linking the variable regions of the heavy (V_{H}) and light (V_{L}) chains of the immunoglobulin with a short linker peptide. Similarly, a disulfide-linked Fv antibody can be generated by linking the V_{H} and V_{L} using an interdomain disulfide bond. On the other hand, sdAbs consist of only the variable region from either the heavy or light chain and usually are the smallest antigen-binding fragments of antibodies. A diabody is a dimer of scFv fragment that consists of the V_{H} and V_{L} regions noncovalent connected by a small peptide linker or covalently linked to each other. The antibodies disclosed herein, including those that comprise an immunogenically active portion of an immunoglobulin molecule, retain the ability to bind a specific antigen.

The term “antigen” refers to an immunogenic molecule that provokes an immune response. This immune response may involve antibody production, activation of specific immunologically competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can also be produced by cells that have been modified or genetically engineered to express an antigen.

The term “codon-optimized” or “codon optimization” when referring to a nucleotide sequence is based on the discovery that the frequency of occurrence of synonymous codons (i.e., codons that code for the same amino acid) in coding nucleotide is biased in different species. Such codon degeneracy allows an identical polypeptide to be encoded by a variety of nucleotide sequences. Codon optimization refers to the process of substituting certain codons in a coding nucleotide sequence with synonymous codons based on the host cell's preference without changing the resulting polypeptide sequence. A variety of codon optimization methods are known in the art, and include, for example, methods disclosed in at least U.S. Pat. Nos. 5,786,464 and 6,114,148.

The term “complementarity-determining regions (CDRs)” is synonymous with “hypervariable region” or “HVR,” and is known in the art to refer to sequences of amino acids within antibody variable regions, which, in general, confer antigen specificity and/or binding affinity and are separated from one another in primary structure by framework sequence. In some cases, framework amino acids can also contribute to binding. In general, there are three CDRs in each variable region. Variable region sequences can be aligned to a numbering scheme (e.g., Kabat, EU, international ImMunoGeneTics Information System® (IMGT®), and AHo), which can allow equivalent residue positions to be annotated and for different molecules to be compared using the Antibody Numbering and Antigen Receptor Classification (ANARCI) software tool (2016, Bioinformatics 15:298-300).

The term “construct” refers to any polynucleotide that contains a recombinant nucleic acid molecule. A construct may be present in a vector (e.g., a bacterial vector or a viral vector) or may be integrated into a genome. A “vector” is a nucleic acid molecule that is capable of introducing a specific nucleic acid sequence into a cell or into another nucleic acid sequence, or servers as a means of transporting another nucleic acid molecule. Vectors may be, for example, plasm ids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vectors), capable of delivering a polynucleotide to a cell genome (e.g., viral vectors), or capable of expressing nucleic acid molecules to which they are linked (expression vectors).

The term “epitope” includes any molecule, structure, amino acid sequence, or protein determinant that is recognized and specifically bound by a cognate binding molecule, such as an antibody or a T-cell receptor, or other binding molecule, domain, or protein.

The term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “host cell” as used herein refers to a cell or microorganism targeted for genetic modification by introduction of a construct or vector carrying a nucleotide sequence for expression of a protein or polypeptide of interest.

The term “nucleic acid” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA) (which includes cDNA, genomic DNA, and synthetic DNA), either of which may be single- or double-stranded. A nucleic acid molecule encoding an amino acid sequence includes all nucleotide sequences that encode the same amino acid sequence.

The term “operably linked” refers to the association of two or more nucleic acid molecules on a single nucleic acid fragment so that the function of one is affected by the other.

The terms “prevent,” “preventing,” and “prevention” as used herein with regard to cancer refer to preventing the onset of the cancer if none had previously occurred, preventing the cancer from occurring in a subject who may be predisposed to the cancer but has not yet been diagnosed as having the cancer, or preventing further cancer development if already present.

The term “subject” refers to a mammalian subject, preferably a human. A “subject in need thereof” refers to a subject who has been diagnosed with cancer or is at an elevated risk of developing cancer. The phrases “subject” and “patient” are used interchangeably herein.

A “therapeutically effective amount” as used herein is an amount that produces a desired effect in a subject for treating a disease. In certain embodiments, the therapeutically effective amount is an amount that yields the maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields the maximum therapeutic effect. A therapeutically effective amount for a particular composition will vary based on a variety of factors, including, but not limited to, the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the host cell, or the pharmaceutical composition containing the same, and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, NY, 2011, the entire disclosures of which are incorporated by reference herein.

The terms “treat,” “treating,” and “treatment” as used herein with regard to cancer refer to alleviating the cancer partially or entirely, preventing the cancer, decreasing the likelihood of occurrence or recurrence of the cancer, slowing the progression or development of the cancer, or eliminating, reducing, or slowing the development of one or more symptoms associated with the cancer. For example, “treating” may refer to preventing or slowing the existing tumor from growing larger, preventing or slowing the formation or metastasis of cancer, and/or slowing the development of certain symptoms of the cancer. In some embodiments, the term “treat,” “treating,” or “treatment” means that the subject has a reduced number of tumors or size of tumor compared to a subject who has not received the treatment. In some embodiments, the term “treat,” “treating,” or “treatment” means that one or more symptoms of the cancer are alleviated in a subject receiving the treatment as disclosed and described herein and/or other cancer therapies comparing to a subject who does not receive such treatment.

The term “variable region” refers to a portion of an antibody heavy or light chain that is involved in antigen binding. Variable regions of antibody heavy (V_{H}) and light (V_{L}) chains each generally comprise four generally conserved framework regions (FRs) and three complementarity-determining regions (CDRs). Framework regions separate CDRs, such that CDRs are situated between framework regions.

The term “vector” refers to a DNA construct containing a nucleic acid molecule that is operably linked to a suitable control sequence capable of effecting the expression of the nucleic acid molecule in a suitable host. Such control sequences may include a promoter to effect transcription, an optional operator sequence to control such transcription, a sequence encoding suitable mRNA ribosome binding sites, and sequences that control termination of transcription and translation. The vector may be a plasmid, a phage particle, a virus, or simply a potential genomic insert. Once transformed into a suitable host, the vector may replicate and function independently of the host genome or may, in some instances, integrate into the genome itself.

### Anti-TSPAN3 Antibodies and Compositions Thereof

In some aspects, provided herein are antibodies that specifically recognize and/or bind to TSPAN3, for example, human TSPAN3. In some embodiments, the antibody is a human antibody. In some embodiments, the antibody is derived from a non-human mammal, for example, a murine antibody, a rat antibody, a rabbit antibody, an equine antibody, or a primate antibody. In some embodiments, the antibody is a monoclonal antibody (mAb).

In some embodiments, the antibody is an inhibitory antibody of TSPAN3, e.g., one that can bind to and inhibit the activity and/or function of TSPAN3. TSPAN3 is a target of the RNA binding protein Musashi 2 and is an important regulator in the oncogenic development and propagation of cancers such as leukemia and several types of solid cancers. As disclosed and described herein, antibodies that specifically bind to TSPAN3 on the surface of cancer cells and inhibit its activity present great potential for the development of novel cancer therapeutics, especially for cancers dependent on the TSPAN3 pathway, including leukemia and solid cancers.

Exemplary sequences of anti-TSPAN3 antibodies and components thereof are provided in Table 1 below. In some embodiments, the anti-TSPAN3 antibody comprises or consists of an amino acid sequence set forth in SEQ ID NO: 2 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 2. In some embodiments, the anti-TSPAN3 antibody comprises or consists of an amino acid sequence set forth in SEQ ID NO: 17 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 17. In some embodiments, the anti-TSPAN3 antibody comprises a heavy chain variable region comprising or consisting of an amino acid sequence set forth in SEQ ID NO: 2 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 2, and/or a light chain variable region comprising or consisting of an amino acid sequence set forth in SEQ ID NO: 17 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 17.

In some embodiments, the anti-TSPAN3 antibody comprises one or more (e.g., one, two, three, four, five, or six) CDRs having amino acid sequences set forth in SEQ ID NOs: 3-5 and 18-20. In some embodiments, the anti-TSPAN3 antibody comprises a heavy chain comprising one or more (e.g., one, two, or three) CDRs having amino acid sequences set forth in SEQ ID NOs: 3-5. In some embodiments, the anti-TSPAN3 antibody comprises a light chain comprising one or more (e.g., one, two, or three) CDRs having amino acid sequences set forth in SEQ ID NOs: 18-20.

In some embodiments, the anti-TSPAN3 antibody comprises or consists of an amino acid sequence set forth in SEQ ID NO: 22 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 22. In some embodiments, the anti-TSPAN3 antibody comprises a light chain comprising one or more (e.g., one, two, or three) CDRs having amino acid sequences set forth in SEQ ID NOs: 23-25.

In some embodiments, the anti-TSPAN3 antibody comprises or consists of an amino acid sequence set forth in SEQ ID NO: 7 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 7. In some embodiments, the anti-TSPAN3 antibody comprises or consists of an amino acid sequence set forth in SEQ ID NO: 27 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 27. In some embodiments, the anti-TSPAN3 antibody comprises a heavy chain variable region comprising or consisting of an amino acid sequence set forth in SEQ ID NO: 7 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 7, and/or a light chain variable region comprising or consisting of an amino acid sequence set forth in SEQ ID NO: 27 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 27.

In some embodiments, the anti-TSPAN3 antibody comprises one or more (e.g., one, two, three, four, five, or six) CDRs having amino acid sequences set forth in SEQ ID NOs: 8-10 and 28-30. In some embodiments, the anti-TSPAN3 antibody comprises a heavy chain comprising one or more (e.g., one, two, or three) CDRs having amino acid sequences set forth in SEQ ID NOs: 8-10. In some embodiments, the anti-TSPAN3 antibody comprises a light chain comprising one or more (e.g., one, two, or three) CDRs having amino acid sequences set forth in SEQ ID NOs: 28-30.

In some embodiments, the anti-TSPAN3 antibody comprises or consists of an amino acid sequence set forth in SEQ ID NO: 12 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 12. In some embodiments, the anti-TSPAN3 antibody comprises or consists of an amino acid sequence set forth in SEQ ID NO: 32 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 32. In some embodiments, the anti-TSPAN3 antibody comprises a heavy chain variable region comprising or consisting of an amino acid sequence set forth in SEQ ID NO: 12 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 12, and/or a light chain variable region comprising or consisting of an amino acid sequence set forth in SEQ ID NO: 32 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 32.

In some embodiments, the anti-TSPAN3 antibody comprises one or more (e.g., one, two, three, four, five, or six) CDRs having amino acid sequences set forth in SEQ ID NOs: 13-15 and 33-35. In some embodiments, the anti-TSPAN3 antibody comprises a heavy chain comprising one or more (e.g., one, two, or three) CDRs having amino acid sequences set forth in SEQ ID NOs: 13-15. In some embodiments, the anti-TSPAN3 antibody comprises a light chain comprising one or more (e.g., one, two, or three) CDRs having amino acid sequences set forth in SEQ ID NOs: 33-35.

In some embodiments, the anti-TSPAN3 antibody comprises a heavy chain variable region comprising or consisting of an amino acid sequence set forth in SEQ ID NO: 2, 7, or 12 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 2, 7, or 12. In some embodiments, the anti-TSPAN3 antibody comprises a light chain variable region comprising or consisting of an amino acid sequence set forth in SEQ ID NO: 17, 22, 27, or 32 or an amino acid sequence that is at least 70% identical (e.g., at least 70%, 75%, 80%, 85%, 90%, 95%, 96%, 97%, 98%, or 99% identical) to the amino acid sequence set forth in SEQ ID NO: 17, 22, 27, or 32. In any of these embodiments, any one of the anti-TSPAN3 antibody heavy chains disclosed can be combined with any one of the anti-TSPAN3 antibody light chains disclosed.

In some embodiments, the anti-TSPAN3 antibody comprises at least one CDR having an amino acid sequence selected from the group consisting of SEQ ID NOs: 3, 4, 5, 8, 9, 10, 13, 14, 15, 18, 19, 20, 23, 24, 25, 28, 29, 30, 33, 34, and 35.

In some embodiments, the anti-TSPAN3 antibody comprises at least two CDRs, each having an amino acid sequence selected from the group consisting of SEQ ID NOs: 3, 4, 5, 8, 9, 10, 13, 14, 15, 18, 19, 20, 23, 24, 25, 28, 29, 30, 33, 34, and 35.

In some embodiments, the anti-TSPAN3 antibody comprises at least three CDRs, each having an amino acid sequence selected from the group consisting of SEQ ID NOs: 3, 4, 5, 8, 9, 10, 13, 14, 15, 18, 19, 20, 23, 24, 25, 28, 29, 30, 33, 34, and 35.

In some embodiments, the anti-TSPAN3 antibody comprises at least four CDRs, each having an amino acid sequence selected from the group consisting of SEQ ID NOs: 3, 4, 5, 8, 9, 10, 13, 14, 15, 18, 19, 20, 23, 24, 25, 28, 29, 30, 33, 34, and 35.

In some embodiments, the anti-TSPAN3 antibody comprises at least five CDRs, each having an amino acid sequence selected from the group consisting of SEQ ID NOs: 3, 4, 5, 8, 9, 10, 13, 14, 15, 18, 19, 20, 23, 24, 25, 28, 29, 30, 33, 34, and 35.

In some embodiments, the anti-TSPAN3 antibody comprises at least six CDRs, each having an amino acid sequence selected from the group consisting of SEQ ID NOs: 3, 4, 5, 8, 9, 10, 13, 14, 15, 18, 19, 20, 23, 24, 25, 28, 29, 30, 33, 34, and 35.

In some embodiments, the anti-TSPAN3 antibody comprises a heavy chain variable region comprising at least one CDR having an amino acid sequence selected from the group consisting of SEQ ID NOs: 3, 4, 5, 8, 9, 10, 13, 14, and 15.

In some embodiments, the anti-TSPAN3 antibody comprises a heavy chain variable region comprising at least two CDRs, each having an amino acid sequence selected from the group consisting of SEQ ID NOs: 3, 4, 5, 8, 9, 10, 13, 14, and 15.

In some embodiments, the anti-TSPAN3 antibody comprises a heavy chain variable region comprising at least three CDRs, each having an amino acid sequence selected from the group consisting of SEQ ID NOs: 3, 4, 5, 8, 9, 10, 13, 14, and 15.

In some embodiments, the anti-TSPAN3 antibody comprises a light chain variable region comprising at least one CDR having an amino acid sequence selected from the group consisting of SEQ ID NOs: 18, 19, 20, 23, 24, 25, 28, 29, 30, 33, 34, and 35.

In some embodiments, the anti-TSPAN3 antibody comprises a light chain variable region comprising at least two CDRs, each having an amino acid sequence selected from the group consisting of SEQ ID NOs: 18, 19, 20, 23, 24, 25, 28, 29, 30, 33, 34, and 35.

In some embodiments, the anti-TSPAN3 antibody comprises a light chain variable region comprising at least three CDRs, each having an amino acid sequence selected from the group consisting of SEQ ID NOs: 18, 19, 20, 23, 24, 25, 28, 29, 30, 33, 34, and 35.

TABLE 1
Exemplary sequences of anti-TSPAN3 antibodies and components

| SEQ ID NO: | Sequence | Description |
|---|---|---|
| 1 | caggtccaactgcagcagcctggggctgag | Anti-TSPAN3 M1 clone |
| | ctggtaaagcctggggcttcagtgaagttgtc | heavy chain variable region |
| | ctgcaaggcttctggctacactttcaccagct | DNA sequence |
| | actggatgcactgggtgaagcagaggcctg | |
| | gacaaggccttgagtggattggaatgattcat | |
| | cctaatagtggtagtcctaactacaatgaga | |
| | ggttcaagagcaaggccacactgactgtag | |
| | acaaatcctccagcacagcctacatgcaact | |
| | cagcagcctgacatctgaggactctgcggtc | |
| | tatttctgtgcaagatggggtgatggttaccac | |
| | aggtacttcgatgtctggggcacagggacca | |
| | cggtcaccgtctcctca | |
| 2 | QVQLQQPGAELVKPGASVKLSC | Anti-TSPAN3 M1 clone |
| | KASGYTFTSYWMHWVKQRPGQ | heavy chain variable region |
| | GLEWIGMIHPNSGSPNYNERFK | amino acid sequence |
| | SKATLTVDKSSSTAYMQLSSLTS | |
| | EDSAVYFCARWGDGYHRYFDV | |
| | WGTGTTVTVSS | |
| 3 | GYTFTSYW | Anti-TSPAN3 M1 clone |
| | | heavy chain CDR1 amino |
| | | acid sequence |
| 4 | IHPNSGSP | Anti-TSPAN3 M1 clone |
| | | heavy chain CDR2 amino |
| | | acid sequence |
| 5 | ARWGDGYHRYFDV | Anti-TSPAN3 M1 clone |
| | | heavy chain CDR3 amino |
| | | acid sequence |
| 6 | cagatccagttggtacagtctggacctgagct | Anti-TSPAN3 M3 clone |
| | gaagaagcctggagagacagtcaagatct | heavy chain variable region |
| | cctgcaaggcttctgggtataccttcacaacc | DNA sequence |
| | tatggaatgagctgggtgaaacaggctcca | |
| | ggaaagggtttaaagtggatgggctggata | |
| | aacacctactctggagtgccaacatatgctg | |
| | atgacttcaagggacggtttgccttctctttgga | |
| | aacctctgccagcactgcctatttgcagatca | |
| | acaacctcaaaaatgaggacacggctacat | |
| | atttctgtgcaagatcacattatagtaacttttat | |
| | gctatggactactggggtcaagaacctcagt | |
| | cacc | |
| 7 | QIQLVQSGPELKKPGETVKISCK | Anti-TSPAN3 M3 clone |
| | ASGYTFTTYGMSWVKQAPGKG | heavy chain variable region |
| | LKWMGWINTYSGVPTYADDFK | amino acid sequence |
| | GRFAFSLETSASTAYLQINNLKN | |
| | EDTATYFCARSHYSNFYAMDY | |
| | WGQEPQSP | |
| 8 | GYTFTTYG | Anti-TSPAN3 M3 clone |
| | | heavy chain CDR1 amino |
| | | acid sequence |
| 9 | INTYSGVP | Anti-TSPAN3 M3 clone |
| | | heavy chain CDR2 amino |
| | | acid sequence |
| 10 | ARSHYSNFYAMDY | Anti-TSPAN3 M3 clone |
| | | heavy chain CDR3 amino |
| | | acid sequence |
| 11 | gaggtccagctgcaacagtctggacctgag | Anti-TSPAN3 M5/M6 clone |
| | ctggtgaagcctggggcttcagtgaagatgt | heavy chain variable region |
| | cctgcaaggcttctggatacacattcactgac | DNA sequence |
| | ttctacatgcattgggtgaagcagagtcatgg | |
| | aaggagccttgagtggattggatatatttacc | |
| | ctcacaatggttttactaagtacaaccagagtt | |
| | tcatgggcaaggccacattgactgtgaacaa | |
| | gtcctccaacacagcctacatggagctccgc | |
| | agcctgacatcggaggattctgcagtctatta | |
| | ctgtgcaagacgttacggggactactggggt | |
| | caaggaatctcagtcaccgtctcctcag | |
| 12 | EVQLQQSGPELVKPGASVKMS | Anti-TSPAN3 M5/M6 clone |
| | CKASGYTFTDFYMHWVKQSHG | heavy chain variable region |
| | RSLEWIGYIYPHNGFTKYNQSF | amino acid sequence |
| | MGKATLTVNKSSNTAYMELRSL | |
| | TSEDSAVYYCARRYGDYWGQG | |
| | ISVTVSS | |
| 13 | GYTFTDFY | Anti-TSPAN3 M5/M6 clone |
| | | heavy chain CDR1 amino |
| | | acid sequence |
| 14 | IYPHNGFT | Anti-TSPAN3 M5/M6 clone |
| | | heavy chain CDR2 amino |
| | | acid sequence |
| 15 | ARRYGDY | Anti-TSPAN3 M5/M6 clone |
| | | heavy chain CDR3 amino |
| | | acid sequence |
| 16 | gacatccagatgactcagtctccagcctccct | Anti-TSPAN3 M1 clone |
| | atctgcatctgtgggagaaactgtcaccatca | light chain variable region |
| | catgtcgagcaagtgggaatattcacaattat | DNA sequence |
| | ttagcatggtatcagcagaaacagggaaaa | |
| | tctcctcagctcctggtctataatgcaaaaac | |
| | cttagcagatggtgtgccatcaaggttcagtg | |
| | gcagtggatcaggaacacaatattctctcaa | |
| | gatcaacagcctgcagcctgaagattttggg | |
| | agttattactgtcaacatttttggagtactcctcc | |
| | gtacacgttcggaggggggaccaagctgg | |
| | aaataaaac | |
| 17 | DIQMTQSPASLSASVGETVTITC | Anti-TSPAN3 M1 clone |
| | RASGNIHNYLAWYQQKQGKSP | light chain variable region |
| | QLLVYNAKTLADGVPSRFSGSG | amino acid sequence |
| | SGTQYSLKINSLQPEDFGSYYC | |
| | QHFWSTPPYTFGGGTKLEIK | |
| 18 | GNIHNY | Anti-TSPAN3 M1 clone |
| | | light chain CDR1 amino |
| | | acid sequence |
| 19 | NAK | Anti-TSPAN3 M1 clone |
| | | light chain CDR2 amino |
| | | acid sequence |
| 20 | QHFWSTPPYT | Anti-TSPAN3 M1 clone |
| | | light chain CDR3 amino |
| | | acid sequence |
| 21 | gaaattgtgctcactcagtctccagccatcac | Anti-TSPAN3 M2 clone |
| | agctgcatctctggggcaaaaggtcaccatc | light chain variable region |
| | acctgcagtgccagctcaagtgtaagttacat | DNA sequence |
| | gaactggtaccagcagaagtcaggcacctc | |
| | ccccaaaccatggatttatgaaatatccaaa | |
| | ctggcttctggagtcccagttcgcttcagtggc | |
| | agtgggtctgggacctcttactctctcacaatc | |
| | agcagcatggaggctgaagatgctgccattt | |
| | attactgccagcagtggaattatcctcttatca | |
| | cgttcggaggggggaccaagctggaaata | |
| | aaac | |
| 22 | EIVLTQSPAITAASLGQKVTITCS | Anti-TSPAN3 M2 clone |
| | ASSSVSYMNWYQQKSGTSPKP | light chain variable region |
| | WIYEISKLASGVPVRFSGSGSGT | amino acid sequence |
| | SYSLTISSMEAEDAAIYYCQQW | |
| | NYPLITFGGGTKLEIK | |
| 23 | SSVSY | Anti-TSPAN3 M2 clone |
| | | light chain CDR1 amino |
| | | acid sequence |
| 24 | EIS | Anti-TSPAN3 M2 clone |
| | | light chain CDR2 amino |
| | | acid sequence |
| 25 | QQWNYPLIT | Anti-TSPAN3 M2 clone |
| | | light chain CDR3 amino |
| | | acid sequence |
| 26 | gacattgtgatgacccagtctcacaaattcat | Anti-TSPAN3 M3 clone |
| | gtccacatcagtaggagacagggtcagcat | light chain variable region |
| | cacctgcaaggccagtcaggatgtgggtact | DNA sequence |
| | gctgtagcctggtatcaacagaaaccaggg | |
| | caatctcctaaactactgatttactgggcatcc | |
| | acccggcacactggagtccctgatcgcttca | |
| | caggcagtggatctgggacagatttcactctc | |
| | accattagcaatgtgcagtctgaagacttggc | |
| | agattatttctgtcagcaatatagcagctatcc | |
| | gctcacgttcggtgctgggaccaagctggag | |
| | ctgaaac | |
| 27 | DIVMTQSHKFMSTSVGDRVSITC | Anti-TSPAN3 M3 clone |
| | KASQDVGTAVAWYQQKPGQSP | light chain variable region |
| | KLLIYWASTRHTGVPDRFTGSG | amino acid sequence |
| | SGTDFTLTISNVQSEDLADYFCQ | |
| | QYSSYPLTFGAGTKLELK | |
| 28 | QDVGTA | Anti-TSPAN3 M3 clone |
| | | light chain CDR1 amino |
| | | acid sequence |
| 29 | WAS | Anti-TSPAN3 M3 clone |
| | | light chain CDR2 amino |
| | | acid sequence |
| 30 | QQYSSYPLT | Anti-TSPAN3 M3 clone |
| | | light chain CDR3 amino |
| | | acid sequence |
| 31 | gacattgtgctgacccaatctccagcttctttg | Anti-TSPAN3 M5/M6 clone |
| | gctgtgtctctggggcagagggccaccatct | light chain variable region |
| | cctgcagagccagcgaaagtcttggtaattat | DNA sequence |
| | ggcattagctatatgaactggttccaacaga | |
| | aaccaggacagccacccaaactcctcatct | |
| | atgctgcatccaaccaaggatccggggtcc | |
| | ctgtcaggtttagtggcagtgggtccgggac | |
| | agacttcagcctcaacatccatcctatggag | |
| | gaggatgatactgcaatgtatttctgtcagca | |
| | aagtaaggaagttccgtggacgttcggtgga | |
| | ggcaccaagctggaaatcaaac | |
| 32 | DIVLTQSPASLAVSLGQRATISC | Anti-TSPAN3 M5/M6 clone |
| | RASESLGNYGISYMNWFQQKP | light chain variable region |
| | GQPPKLLIYAASNQGSGVPVRF | amino acid sequence |
| | SGSGSGTDFSLNIHPMEEDDTA | |
| | MYFCQQSKEVPWTFGGGTKLEI | |
| | K | |
| 33 | ESLGNYGISY | Anti-TSPAN3 M5/M6 clone |
| | | light chain CDR1 amino |
| | | acid sequence |
| 34 | AAS | Anti-TSPAN3 M5/M6 clone |
| | | light chain CDR2 amino |
| | | acid sequence |
| 35 | QQSKEVPWT | Anti-TSPAN3 M5/M6 clone |
| | | light chain CDR3 amino |
| | | acid sequence |

In some embodiments, the anti-TSPAN3 antibody according to various embodiments disclosed herein is a single-chain variable fragment antibody (scFv). An scFv antibody may be generated by linking the variable regions of the heavy and light chains as described with a short linker peptide. In some embodiments, the anti-TSPAN3 antibody is a disulfide-linked Fv antibody, which can be generated by linking the variable regions of the heavy and light chains as described using an interdomain disulfide bond. In some embodiments, the anti-TSPAN3 antibody is a single-domain antibody (sdAb), which usually consists of only the variable region from either the heavy or light chain.

In some embodiments, the anti-TSPAN3 antibody according to various embodiments disclosed herein is a humanized antibody. A “humanized” form of a non-human (e.g., rodent) antibody is a chimeric antibody including a sequence derived from a non-human antibody. In many cases, a humanized antibody is a human immunoglobulin in which a residue in a variable region of a subject species is replaced with a residue in a variable region of a non-human species (donor antibody) having a desired specificity, affinity, and ability, for example, a mouse, a rat, a rabbit, or a non-human primate. In some cases, a residue in a framework region (FR) of the human immunoglobulin is replaced with a corresponding non-human residue. In addition, a humanized antibody may include a residue that is not found in a recipient or donor antibody. In some embodiments, such modification may be performed to further improve antibody performance. Techniques used to humanize a monoclonal antibody are within the purview of one of ordinary skill in the art.

In some aspects, provided herein are pharmaceutical compositions comprising an antibody that specifically binds to TSPAN3 according to various embodiments disclosed and described herein. The pharmaceutical compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Non-limiting examples of such carriers or excipients include, but are not limited to, water, saline, Ringer's solutions, dextrose solution, and 5% human serum albumin. Liposomes and non-aqueous vehicles such as fixed oils may also be used.

In some embodiments, the pharmaceutical compositions can be formulated (e.g., injectable, lyophilized, liquid formulations, or oral formulations) to be compatible with their intended route of administration. Examples of routes of administration include oral, parenteral, intravenous, intradermal, subcutaneous, and rectal administration. Solutions or suspensions used for parenteral, intradermal, or subcutaneous application can include the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates, or phosphates; and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, or multiple-dose vials made of glass or plastic.

In some embodiments, the pharmaceutical compositions can be co-formulated in the same dosage unit or can be individually formulated in separate dosage units. The term “dosage unit” herein refers to a portion of a pharmaceutical composition that contains an amount of a therapeutic agent suitable fora single administration to provide a therapeutic effect. Such dosage units may be administered one to a plurality (e.g., 1 to about 10, 1 to 8, 1 to 6, 1 to 4, or 1 to 2) of times per day, or as many times as needed to elicit a therapeutic response.

### Nucleic Acid Sequences, Vectors, Host Cells, and Compositions Thereof

In some aspects, provided herein are nucleic acid molecules or polynucleotides comprising a nucleotide sequence that encodes an antibody that specifically binds to TSPAN3 according to various embodiments disclosed herein. The nucleic acids or polynucleotides may be used for production of recombinant anti-TSPAN3 antibodies in a host cell.

In some embodiments, the nucleic acid comprises or consists of a nucleotide sequence set forth in any of SEQ ID NOs: 1, 6, 11, 16, 21, 26, and 31 or a nucleotide sequence that is at least 70% identical (e.g., at least 70%, 75%, 80%, 85%, 90%, 95%, 96%, 97%, 98%, or 99% identical) to the nucleotide sequence set forth in any of SEQ ID NOs: 1, 6, 11, 16, 21, 26, and 31.

In some embodiments, the nucleotide sequence encoding an anti-TSPAN3 antibody may be codon-optimized for expression in a host cell, for example, a mammalian cell, using known techniques in the art. Codon-optimized sequences include sequences that are partially or fully codon-optimized.

In some embodiments, the nucleotide sequence encoding an anti-TSPAN3 antibody may be inserted into a vector for delivery and expression in a host cell. The vector can be any type of vector suitable for introduction of nucleotide sequences into a host cell, including, for example, plasmids, adenoviral vectors, adenoviral associated vectors, retroviral vectors, lentiviral vectors, and phages. In certain of these embodiments, the vector is one that facilitates the integration of the nucleotide sequence into a host cell's genome upon introduction into the host cell and thereby replication of the nucleotide sequence along with the host genome, such as a viral vector. Viral vectors include retroviruses, adenoviruses, parvoviruses, coronaviruses, negative strand RNA viruses, positive strand RNA viruses, and double-stranded DNA viruses.

In some embodiments, the nucleotide sequence encoding an anti-TSPAN3 antibody may be operatively linked to certain regulatory elements of the vector. As known to a skilled artisan, expression vectors are typically engineered to contain polynucleotide sequences that are needed to affect the expression and processing of coding sequences to which they are operatively linked. Expression control sequences may include appropriate transcription initiation, termination, promoter, and enhancer sequences; efficient RNA processing signals, such as splicing and polyadenylation signals; sequences that stabilize cytoplasmic mRNA; sequences that enhance translation efficiency; sequences that enhance protein stability; and possibly sequences that enhance protein secretion. Expression control sequences may be operatively linked if they are contiguous with the gene of interest and expression control sequences that act in trans or at a distance to control the gene of interest.

In some embodiments, the promoter is one that drives constitutive gene expression in mammalian cells. Those frequently used include, for example, elongation factor 1 alpha (EF1α) promoter, cytomegalovirus (CMV) immediate-early promoter (Greenaway et al., Gene 18: 355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60(1):65-74 (1987)), human beta actin promoter, polyubiquitin C gene (UBC) promoter, and CAG promoter (Nitoshi et al., Gene 108:193-199 (1991)). In some embodiments, the promoter is an inducible promoter. Unlike constitutive promoters, inducible promoters can switch between an on and an off state in response to certain stimuli (e.g., chemical agents, temperature, and light) and can be regulated in tissue- or cell-specific manners.

In some embodiments, the vector according to various embodiments disclosed herein may be present in a composition. In some embodiments, the composition may further comprise one or more pharmaceutically acceptable carriers, excipients, preservatives, or a combination thereof.

In some aspects, provided herein are host cells that contain the nucleotide sequence encoding an anti-TSPAN3 antibody according to various embodiments disclosed herein. The nucleotide sequence encoding an anti-TSPAN3 antibody of the present technology, or vectors containing the same, can be used to transfect or transduce a host cell so that the host cell may express or produce the anti-TSPAN3 antibody. Host cells may be induced to incorporate the nucleotide sequence by transformation via calcium phosphate precipitation, DEAE-dextran, electroporation, microinjection, or other methods.

In some embodiments, the host cell is a prokaryotic cell, for example, a bacterial cell. In some embodiments, the host cell is a eukaryotic cell, for example, a yeast cell, an insect cell, or a mammalian cell. In some embodiments, the host cell is a mammalian cell, for example, a human cell.

In some embodiments, the host cell according to various embodiments disclosed herein may be present in a composition. In some embodiments, the composition may further comprise one or more pharmaceutically acceptable carriers, excipients, preservatives, or a combination thereof.

### Methods of Treatment

In some aspects, provided herein are methods for treating and/or preventing cancer in a subject in need thereof. In some embodiments, the cancer is TSPAN3-dependent, e.g., one that expresses TSPAN3 or involves TSPAN3 signaling. The method entails administering to the subject a therapeutically effective amount of an antibody that specifically binds to TSPAN3, a cell that produces the antibody, or a pharmaceutical composition containing the antibody or the cell, according to various embodiments disclosed herein. In certain embodiments, the method further entails administering one or more other cancer therapies such as surgery, immunotherapy, radiotherapy, and/or chemotherapy to the subject sequentially or simultaneously.

In some embodiments, the cancer is a hematologic malignancy. Non-limiting examples of hematologic malignancies include myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

In some embodiments, the cancer is a solid cancer. Non-limiting examples of solid cancers that express TSPAN3 include pancreatic cancer, glioma, glioblastoma, colorectal cancer, thyroid cancer, stomach cancer, ovarian cancer, melanoma, endometrial cancer, lung cancer, renal cancer, cervical cancer, prostate cancer, breast cancer, urothelial cancer, testicular cancer, head and neck cancer, liver cancer, esophageal cancer, and other types of solid cancers that express TSPAN3.

In some embodiments, the method comprises administering an antibody that specifically binds to TSPAN3, or a pharmaceutical composition containing the same, to the subject at an amount of about 10 mg/kg to about 150 mg/kg, from 30 mg/kg to about 120 mg/kg, or from 60 mg/kg to about 90 mg/kg. In some embodiments, the antibody that specifically binds to TSPAN3, or a pharmaceutical composition containing the same, is administered to the subject at an amount of more than about 150 mg/kg. In some embodiments, a therapeutically effective amount of an antibody that specifically binds to TSPAN3, or a pharmaceutical composition containing the same, is about 15 mg/kg, about 30 mg/kg, about 45 mg/kg, about 60 mg/kg, about 75 mg/kg, about 90 mg/kg, about 105 mg/kg, about 120 mg/kg, about 135 mg/kg, about 150 mg/kg, or more than about 150 mg/kg. In some embodiments, a single dose or multiple doses may be administered to a subject. In some embodiments, the antibody that specifically binds to TSPAN3, a cell that produces the antibody, or a pharmaceutical composition containing the antibody or the cell, is administered once or multiple times a day.

In some embodiments, the amount of cells producing the anti-TSPAN3 antibodies administered in a pharmaceutical composition is typically greater than 10² cells, for example, about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰ cells, or more.

In some embodiments, the methods comprise administering to the subject an antibody that specifically binds to TSPAN3, a cell that produces the antibody, or a pharmaceutical composition containing the antibody or the cell, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the antibody or fragment thereof, or the pharmaceutical composition containing the same, can be administered every day, every other day, every third day, weekly, biweekly (i.e., every other week), every third week, monthly, every other month, or every third month.

It is within the purview of one of ordinary skill in the art to select a suitable administration route, such as oral administration, subcutaneous administration, intravenous administration, intramuscular administration, intradermal administration, intrathecal administration, or intraperitoneal administration. For treating a subject in need thereof, the antibody or fragment thereof, or the pharmaceutical composition containing the same, can be administered continuously or intermittently, for an immediate release, controlled release, or sustained release.

In some embodiments, the antibody that specifically binds to TSPAN3, a cell that produces the antibody, or a pharmaceutical composition containing the antibody or the cell, may be administered over a predetermined time period. Alternatively, the antibody that specifically binds to TSPAN3, a cell that produces the antibody, or a pharmaceutical composition containing the antibody or the cell, may be administered until a particular therapeutic benchmark is reached. In some embodiments, the methods provided herein include a step of evaluating one or more therapeutic benchmarks in a biological sample, such as, but not limited to, the level of a cancer biomarker, to determine whether to continue administration of the treatment.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent, a chemotherapy agent, and a biologic agent. In some embodiments, the subject was administered the one or more additional therapeutic agents before administration of the antibody that specifically binds to TSPAN3, a cell that produces the antibody, or a pharmaceutical composition containing the antibody or the cell. In some embodiments, the subject is co-administered the one or more additional therapeutic agents and the antibody that specifically binds to TSPAN3, a cell that produces the antibody, or a pharmaceutical composition containing the antibody or the cell. In some embodiments, the subject was administered the one or more additional therapeutic agents before after administration of the antibody that specifically binds to TSPAN3, a cell that produces the antibody, or a pharmaceutical composition containing the antibody or the cell.

As one of ordinary skill in the art would understand, the one or more additional therapeutic agents and the antibody that specifically binds to TSPAN3, a cell that produces the antibody, or a pharmaceutical composition containing the antibody or the cell, can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combination therapy achieves improved or synergistic effects in comparison to any of the treatments administered alone.

### EXAMPLE

### Example 1. Generation, Characterization, and Testing of TSPAN3 Antibodies

In this study, anti-human TSPAN3 monoclonal antibodies (mAbs) were generated and purified from hybridoma clones and then tested for their inhibitory effects in vitro in human primary leukemia cells and in vivo in xenograft models. The results suggest that the antibodies may be valuable potential therapeutics to treat aggressive myeloid leukemias as well as any cancers that express TSPAN3.

Generation of Anti-Human TSPAN3 mAbs

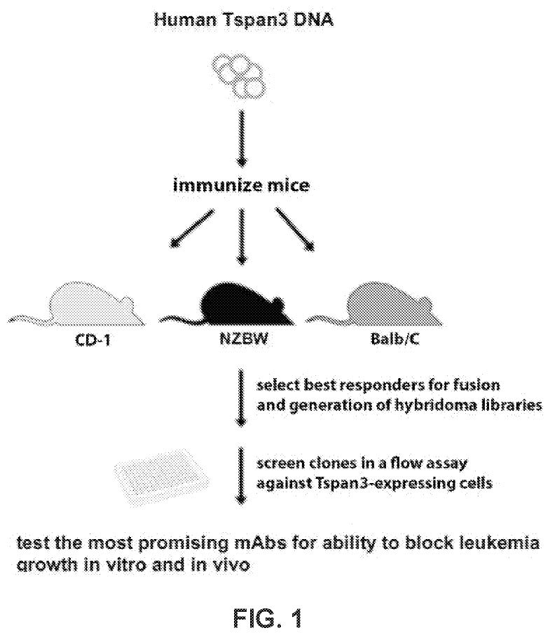

Inhibitory mAbs against human TSPAN3 were developed using a tailored strategy designed to increase antibody diversity and the odds of a positive immune response. A DNA/cell-based approach was used (FIG. 1). Specifically, codon-optimized human Tspan3 DNA was synthesized and used to immunize varying strains of mice. A stable cell line overexpressing human TSPAN3 was also generated by transfection with the same Tspan3 DNA, and the TSPAN3-expressing cells were used to boost the mice. Mice were then immunized with either DNA/adjuvant or TSPAN3-expressing cells over the course of approximately 15 weeks (15 injections of DNA; 2 injections of cells over the course of this time period), after which they received a final boost with TSPAN3-expressing cells. Three distinct cohorts of mice were immunized, with each cohort comprising mice from a different strain (e.g., NZBW, CD-1, Balb/C). Serum from each mouse was tested by fluorescence-activated cell sorting (FACS) at several time points during the immunization protocol for the ability to bind to human TSPAN3-expressing cells. Three of the mice (one of the NZBW-immunized mice and two of the CD-1-immunized mice) showed the strongest immune response by day 92 of the immunization protocol. These results suggested that clones recognizing cell surface-expressed human TSPAN3 are present in these animals. To increase titers across these as well as the other cohorts of mice, the animals were boosted with one additional DNA injection one week later, followed by immunization with TSPAN3-expressing cells after five additional days.

Using the hybridoma technology, i.e., fusing the antibody-producing B-cells harvested from the immunized mice with myeloma cells, two hybridoma fusion libraries were generated three days later (one from the reactive NZBW mouse and one from the two pooled reactive CD-1 mice), and then retested for reactivity to surface-expressed human TSPAN3. Although there was some binding to both the TSPAN3 cells and the parental mouse cells, the TSPAN3 hybridoma library from the CD-1 mice showed the most favorable binding results to pursue clones. Based on these results, the hybridoma library was cloned and screened by FACS to identify individual clones with the ability to bind cell surface expressed TSPAN3. As shown in Table 2, six clones with the binding ability were subsequently identified:

TABLE 2
Flow-based analysis of mAb binding to TSPAN3-expressing cells

| | Median Fluorescence | |
|---|---|---|
| Clone Designation | Intensity (MFI) | % Gated |
| Clone 1 (TSPAN3-C - M1) | 290.07 | 59.03 |
| Clone 2 (TSPAN3-C - M2) | 404.35 | 76.92 |
| Clone 3 (TSPAN3-C - M3) | 283.94 | 60.71 |
| Clone 4 (TSPAN3-C - M4) | 341.55 | 75.88 |
| Clone 5 (TSPAN3-C - M5) | 529.38 | 73.02 |
| Clone 6 (TSPAN3-C - M6) | 307.98 | 69.47 |
| Controls | MFI | % Gated |
| Media | 134.30 | 2.26 |
| Hyb Lib | 348.88 | 59.60 |
| Sera | 156.16 | 13.55 |

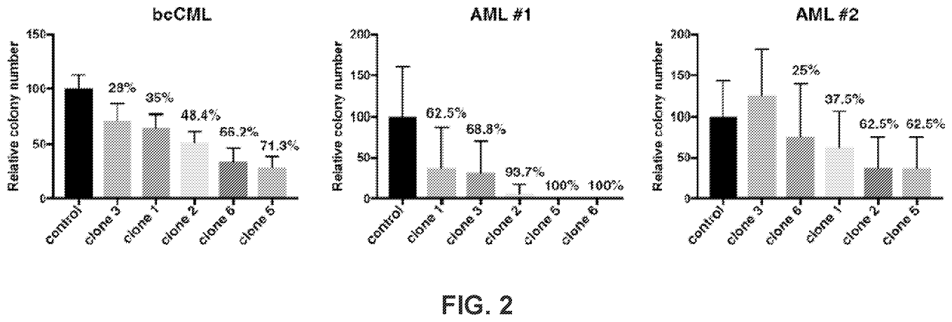

Subsequently, five of the six identified clones (M1, M2, M3, M5, and M6) were tested for their ability to block the growth of primary patient myeloid leukemia samples using the colony assay. M4 did not grow out and thus could not be pursued. FIG. 2 shows colony forming assays of primary patient samples cultured with supernatants from the indicated hybridoma clones. Three independent primary patient samples were tested, including one sample of blast crisis chronic myelogenous leukemia (bcCML) and two samples of acute myelogenous leukemia (AML), as indicated in the figure. Percent inhibition of colony formation relative to control is indicated for each clone. As shown in FIG. 2, supernatants from several of the clones inhibited colony formation in three independent leukemia patient samples. Indeed, the clones showed a range of inhibition of primary patient colony formation, including 75% or more in specific patient samples. For example, clone M6 and clone M5 showed a 66.2% and 71.3% inhibition of colony formation in the bcCML sample, respectively. For another example, clones M2, M5, and M6 showed a respective 93.7%, 100%, and 100% inhibition of colony formation in one of the AML samples.

Subsequently, antibodies were purified from the hybridoma supernatants. Two independent rounds of scale-up production and purification were performed from each hybridoma clone. Germline, isotype, DNA sequence, protein sequence, and amino acid sequence of the complementarity-determining regions (CDRs) of the heavy and light chains for clones M1, M2, M3, M5, and M6 are shown in Table 3:

TABLE 3
Identification of hybridoma clones
Heavy Chain

| Clone | Germline | Isotype | Sequence |
|---|---|---|---|
| M1 | 99.0% (291/294) | IgG1 | M1 heavy chain full variable region DNA sequence*: |
| | IGHV1-64*01 | | caggtccaactgcagcagcctggggctgagctggtaaagcctggggcttcagtgaag |
| | | | ttgtcctgcaaggcttctggctacactttcaccagctactggatgcactgggtgaagcag |
| | | | aggcctggacaaggccttgagtggattggaatgattcatcctaatagtggtagtcctaa |
| | | | ctacaatgagaggttcaagagcaaggccacactgactgtagacaaatcctccagca |
| | | | cagcctacatgcaactcagcagcctgacatctgaggactctgcggtctatttctgtgca |
| | | | agatggggtgatggttaccacaggtacttcgatgtctggggcacagggaccacggtc |
| | | | accgtctcctca (SEQ ID NO: 1) |
| | | | M1 heavy chain full variable region amino acid sequence: |
| | | | QVQLQQPGAELVKPGASVKLSCKASGYTFTSYWMHWVKQR |
| | | | PGQGLEWIGMIHPNSGSPNYNERFKSKATLTVDKSSSTAYM |
| | | | QLSSLTSEDSAVYFCARWGDGYHRYFDWWGTGTTVTVSS |
| | | | (SEQ ID NO: 2) |
| | | | M1 heavy chain CDR1 amino acid sequence: |
| | | | GYTFTSYW (SEQ ID NO: 3) |
| | | | M1 heavy chain CDR2 amino acid sequence: |
| | | | IHPNSGSP (SEQ ID NO: 4) |
| | | | M1 heavy chain CDR3 amino acid sequence: |
| | | | ARWGDGYHRYFDV (SEQ ID NO: 5) |
| M2 | Not yet determined | N/D | N/D |
| | (N/D) | | |
| M3 | 100.0% (294/294) | IgM | M3 heavy chain full variable region DNA sequence*: |
| | IGHV9-3*01 | | cagatccagttggtacagtctggacctgagctgaagaagcctggagagacagtcaa |
| | | | gatctcctgcaaggcttctgggtataccttcacaacctatggaatgagctgggtgaaac |
| | | | aggctccaggaaagggtttaaagtggatgggctggataaacacctactctggagtgc |
| | | | caacatatgctgatgacttcaagggacggtttgccttctctttggaaacctctgccagca |
| | | | ctgcctatttgcagatcaacaacctcaaaaatgaggacacggctacatatttctgtgca |
| | | | agatcacattatagtaacttttatgctatggactactggggtcaagaacctcagtcacc |
| | | | (SEQ ID NO: 6) |
| | | | M3 heavy chain full variable region amino acid sequence: |
| | | | QIQLVQSGPELKKPGETVKISCKASGYTFTTYGMSWVKQAP |
| | | | GKGLKWMGWINTYSGVPTYADDFKGRFAFSLETSASTAYLQI |
| | | | NNLKNEDTATYFCARSHYSNFYAMDYWGQEPQSP (SEQ ID |
| | | | NO: 7) |
| | | | M3 heavy chain CDR1 amino acid sequence: |
| | | | GYTFTTYG (SEQ ID NO: 8) |
| | | | M3 heavy chain CDR2 amino acid sequence: |
| | | | INTYSGVP (SEQ ID NO: 9) |
| | | | M3 heavy chain CDR3 amino acid sequence: |
| | | | ARSHYSNFYAMDY (SEQ ID NO: 10) |
| M5 | 94.6% (278/294) | IgG2b | M5 heavy chain full variable region DNA sequence*: |
| | IGHV1-22*01 | | gaggtccagctgcaacagtctggacctgagctggtgaagcctggggcttcagtgaag |
| | | | atgtcctgcaaggcttctggatacacattcactgacttctacatgcattgggtgaagcag |
| | | | agtcatggaaggagccttgagtggattggatatatttaccctcacaatggttttactaagt |
| | | | acaaccagagtttcatgggcaaggccacattgactgtgaacaagtcctccaacacag |
| | | | cctacatggagctccgcagcctgacatcggaggattctgcagtctattactgtgcaaga |
| | | | cgttacggggactactggggtcaaggaatctcagtcaccgtctcctcag (SEQ ID |
| | | | NO: 11) |
| | | | M5 heavy chain full variable region amino acid sequence: |
| | | | EVQLQQSGPELVKPGASVKMSCKASGYTFTDFYMHWVKQS |
| | | | HGRSLEWIGYIYPHNGFTKYNQSFMGKATLTVNKSSNTAYM |
| | | | ELRSLTSEDSAVYYCARRYGDYWGQGISVTVSS (SEQ ID |
| | | | NO: 12) |
| | | | M5 heavy chain CDR1 amino acid sequence: |
| | | | GYTFTDFY (SEQ ID NO: 13) |
| | | | M5 heavy chain CDR2 amino acid sequence: |
| | | | IYPHNGFT (SEQ ID NO: 14) |
| | | | M5 heavy chain CDR3 amino acid sequence: |
| | | | ARRYGDY (SEQ ID NO: 15) |
| M6 | 87.5% (258/295) | IgG2b | M6 heavy chain full variable region DNA sequence*: |
| | IGHV1-22*01 | | gaggtccagctgcaacagtctggacctgagctggtgaagcctggggcttcagtgaag |
| | | | atgtcctgcaaggcttctggatacacattcactgacttctacatgcactgggtgaagcag |
| | | | agtcatggaaggagccttgagtggattggatatatttaccctcacaatggttttactaagt |
| | | | acaaccagagtttcatgggcaaggccacattgactgtgaacaagtcctccaacacag |
| | | | cctacatggagctccgcagcctgacatcggaggattctgcagtctattactgtgcaaga |
| | | | cgttacggggactactggggtcaaggaatctcagtcaccgtctcctcag (SEQ ID |
| | | | NO: 11) |
| | | | M6 heavy chain full variable region amino acid sequence: |
| | | | EVQLQQSGPELVKPGASVKMSCKASGYTFTDFYMHWVKQS |
| | | | HGRSLEWIGYIYPHNGFTKYNQSFMGKATLTVNKSSNTAYM |
| | | | ELRSLTSEDSAVYYCARRYGDYWGQGISVTVSS (SEQ ID |
| | | | NO: 12) |
| | | | M6 heavy chain CDR1 amino acid sequence: |
| | | | GYTFTDFY (SEQ ID NO: 13) |
| | | | M6 heavy chain CDR2 amino acid sequence: |
| | | | IYPHNGFT (SEQ ID NO: 14) |
| | | | M6 heavy chain CDR3 amino acid sequence: |
| | | | ARRYGDY (SEQ ID NO: 15) |

Light Chain

| Clone | Germline | Isotype | Sequence |
|---|---|---|---|
| M1 | 100.0% (287/287) | KAPPA | M1 light chain full variable region DNA sequence*: |
| | IGKV12-41*01 | | gacatccagatgactcagtctccagcctccctatctgcatctgtgggagaaactgtcac |
| | | | catcacatgtcgagcaagtgggaatattcacaattatttagcatggtatcagcagaaac |
| | | | agggaaaatctcctcagctcctggtctataatgcaaaaaccttagcagatggtgtgcca |
| | | | tcaaggttcagtggcagtggatcaggaacacaatattctctcaagatcaacagcctgc |
| | | | agcctgaagattttgggagttattactgtcaacatttttggagtactcctccgtacacgt |
| | | | gtcgaggggggaccaagctggaaataaaac (SEQ ID NO: 16) |
| | | | M1 light chain full variable region amino acid sequence: |
| | | | DIQMTQSPASLSASVGETVTITCRASGNIHNYLAWYQQKQGK |
| | | | SPQLLVYNAKTLADGVPSRFSGSGSGTQYSLKINSLQPEDFG |
| | | | SYYCQHFWSTPPYTFGGGTKLEIK (SEQ ID NO: 17) |
| | | | M1 light chain CDR1 amino acid sequence: |
| | | | GNIHNY (SEQ ID NO: 18) |
| | | | M1 light chain CDR2 amino acid sequence: |
| | | | NAK (SEQ ID NO: 19) |
| | | | M1 light chain CDR3 amino acid sequence: |
| | | | QHFWSTPPYT (SEQ ID NO: 20) |
| M2 | 99.3% (281/283) | KAPPA | M2 light chain full variable region DNA sequence*: |
| | IGKV4-86*01 | | gaaattgtgctcactcagtctccagccatcacagctgcatctctggggcaaaaggtca |
| | | | ccatcacctgcagtgccagctcaagtgtaagttacatgaactggtaccagcagaagtc |
| | | | aggcacctcccccaaaccatggatttatgaaatatccaaactggcttctggagtccca |
| | | | gttcgcttcagtggcagtgggtctgggacctcttactctctcacaatcagcagcatggag |
| | | | gctgaagatgctgccatttattactgccagcagtggaattatcctcttatcacgttcggag |
| | | | gggggaccaagctggaaataaaac (SEQ ID NO: 21) |
| | | | M2 light chain full variable region amino acid sequence: |
| | | | EIVLTQSPAITAASLGQKVTITCSASSSVSYMNWYQQKSGTS |
| | | | PKPWIYEISKLASGVPVRFSGSGSGTSYSLTISSMEAEDAAIY |
| | | | YCQQWNYPLITFGGGTKLEIK (SEQ ID NO: 22) |
| | | | M2 light chain CDR1 amino acid sequence: |
| | | | SSVSY (SEQ ID NO: 23) |
| | | | M2 light chain CDR2 amino acid sequence: |
| | | | EIS (SEQ ID NO: 24) |
| | | | M2 light chain CDR3 amino acid sequence: |
| | | | QQWNYPLIT (SEQ ID NO: 25) |
| M3 | 99.7% (286/287) | KAPPA | M3 light chain full variable region DNA sequence*: |
| | IGKV6-23*01 | | gacattgtgatgacccagtctcacaaattcatgtccacatcagtaggagacagggtca |
| | | | gcatcacctgcaaggccagtcaggatgtgggtactgctgtagcctggtatcaacagaa |
| | | | accagggcaatctcctaaactactgatttactgggcatccacccggcacactggagtc |
| | | | cctgatcgcttcacaggcagtggatctgggacagatttcactctcaccattagcaatgtg |
| | | | cagtctgaagacttggcagattatttctgtcagcaatatagcagctatccgctcacgttcg |
| | | | gtgctgggaccaagctggagctgaaac (SEQ ID NO: 26) |
| | | | M3 light chain full variable region amino acid sequence: |
| | | | DIVMTQSHKFMSTSVGDRVSITCKASQDVGTAVAWYQQKPG |
| | | | QSPKLLIYWASTRHTGVPDRFTGSGSGTDFTLTISNVQSEDL |
| | | | ADYFCQQYSSYPLTFGAGTKLELK (SEQ ID NO: 27) |
| | | | M3 light chain CDR1 amino acid sequence: |
| | | | QDVGTA (SEQ ID NO: 28) |
| | | | M3 light chain CDR2 amino acid sequence: |
| | | | WAS (SEQ ID NO: 29) |
| | | | M3 light chain CDR3 amino acid sequence: |
| | | | QQYSSYPLT (SEQ ID NO: 30) |
| M5 | 97.2% (242/249) | KAPPA | M5 light chain full variable region DNA sequence*: |
| | IGKV3-2*01 | | gacattgtgctgacccaatctccagcttctttggctgtgtctctggggcagagggccacc |
| | | | atctcctgcagagccagcgaaagtcttggtaattatggcattagctatatgaactggttc |
| | | | caacagaaaccaggacagccacccaaactcctcatctatgctgcatccaaccaagg |
| | | | atccggggtccctgtcaggtttagtggcagtgggtccgggacagacttcagcctcaac |
| | | | atccatcctatggaggaggatgatactgcaatgtatttctgtcagcaaagtaaggaagtt |
| | | | ccgtggacgttcggtggaggcaccaagctggaaatcaaac (SEQ ID NO: 31) |
| | | | M5 light chain full variable region amino acid sequence: |
| | | | DIVLTQSPASLAVSLGQRATISCRASESLGNYGISYMNWFQQ |
| | | | KPGQPPKLLIYAASNQGSGVPVRFSGSGSGTDFSLNIHPMEE |
| | | | DDTAMYFCQQSKEVPWTFGGGTKLEIK (SEQ ID NO: 32) |
| | | | M5 light chain CDR1 amino acid sequence: |
| | | | ESLGNYGISY (SEQ ID NO: 33) |
| | | | M5 light chain CDR2 amino acid sequence: |
| | | | AAS (SEQ ID NO: 34) |
| | | | M5 light chain CDR3 amino acid sequence: |
| | | | QQSKEVPWT (SEQ ID NO: 35) |
| M6 | 96.1% (248/258) | KAPPA | M6 light chain full variable region DNA sequence*: |
| | IGKV3-2*01 | | gacattgtgctgacccaatctccagcttctttggctgtgtctctggggcagagggccacc |
| | | | atctcctgcagagccagcgaaagtcttggtaattatggcattagctatatgaactggttc |
| | | | caacagaaaccaggacagccacccaaactcctcatctatgctgcatccaaccaagg |
| | | | atccggggtccctgtcaggtttagtggcagtgggtccgggacagacttcagcctcaac |
| | | | atccatcctatggaggaggatgatactgcaatgtatttctgtcagcaaagtaaggaagtt |
| | | | ccgtggacgttcggtggaggcaccaagctggaaatcaaac (SEQ ID NO: 31) |
| | | | M6 light chain full variable region amino acid sequence: |
| | | | DIVLTQSPASLAVSLGQRATISCRASESLGNYGISYMNWFQQ |
| | | | KPGQPPKLLIYAASNQGSGVPVRFSGSGSGTDFSLNIHPMEE |
| | | | DDTAMYFCQQSKEVPWTFGGGTKLEIK (SEQ ID NO: 32) |
| | | | M6 light chain CDR1 amino acid sequence: |
| | | | ESLGNYGISY (SEQ ID NO: 33) |
| | | | M6 light chain CDR2 amino acid sequence: |
| | | | AAS (SEQ ID NO: 34) |
| | | | M6 light chain CDR3 amino acid sequence: |
| | | | QQSKEVPWT (SEQ ID NO: 35) |

*Consists of the following segments in order from the N-terminal: Framework 1-CDR1-Framework 2-CDR2-Framework 3-CDR3-Framework 4.

As shown in Table 3, the “Germline” column indicates the gene from which the variable region of the immunoglobulin heavy or light chain of each monoclonal antibody most likely derived. For example, a germ line match such as “IGKV3-2*01” indicates that the light chain variable region of a particular antibody most likely came about from the selection and affinity maturation of the Ig-Kappa Variable Region Gene 3-2*01 in the kappa light chain locus.

### Inhibition of Leukemia Cell Growth In Vitro

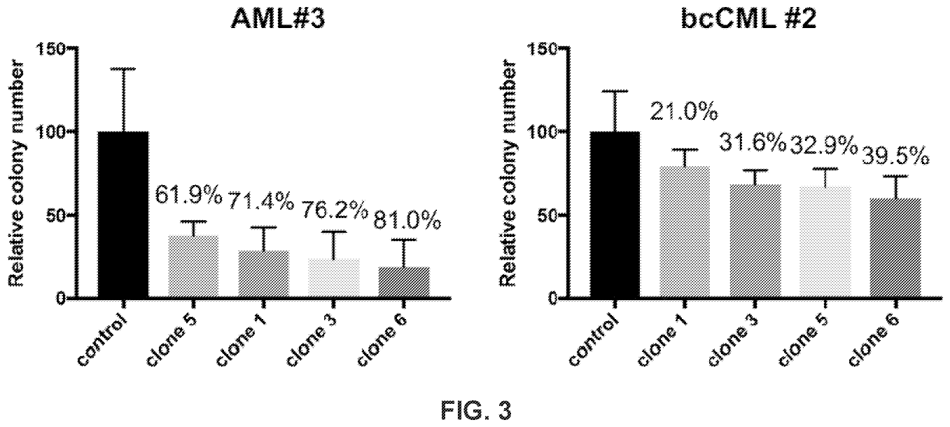

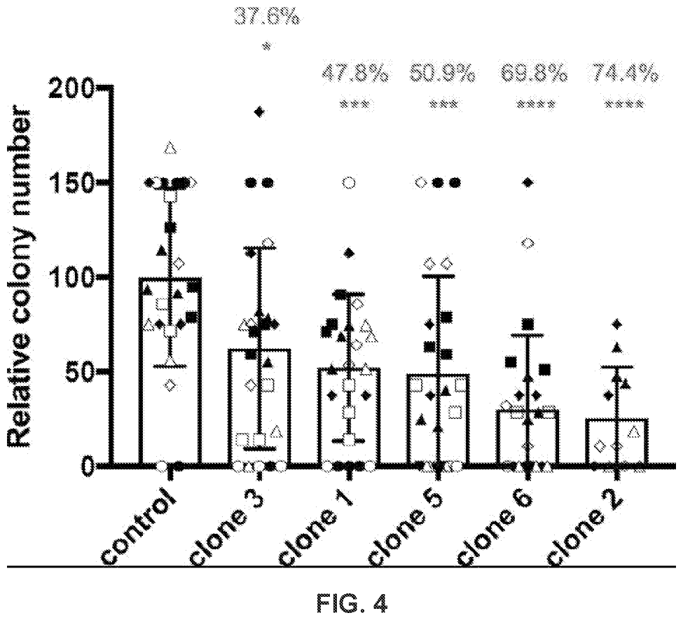

Next, the purified antibodies (5 μg/ml) were tested against multiple primary patient samples, including three independent AML patient samples and two independent bcCML patient samples (FIGS. 3-4). FIG. 3 shows the impact of purified TSPAN3 mAbs on the growth of primary leukemia patient cells in vitro. FIG. 4 is a compilation of data from a total of five independent AML patient samples and three independent bcCML patient samples. As shown in the figures, all of the clones inhibited the colony forming ability of leukemia patient samples to varying degrees, with some showing a deeper impact than others. Notably, some patient samples were more effectively blocked than others; this may have to do with the characteristics of the sample—for example, which oncogenes/mutations they carry, the patient's disease stage, and the level of surface TSPAN3.

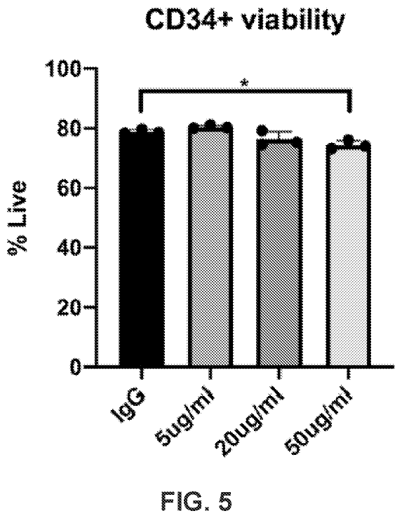

Importantly, viability of normal human CD34+ cells was not reduced in the presence of increasing concentrations of anti-TSPAN3 mAb (FIG. 5). As shown, viability was measured 24 hours after culture with control or varying amount of M6 mAb, and the M6 anti-TSPAN3 mAb did not impair growth of normal human CD34+ hematopoietic cells at any of the amounts tested.

### Inhibition of Leukemia Cell Growth In Vivo

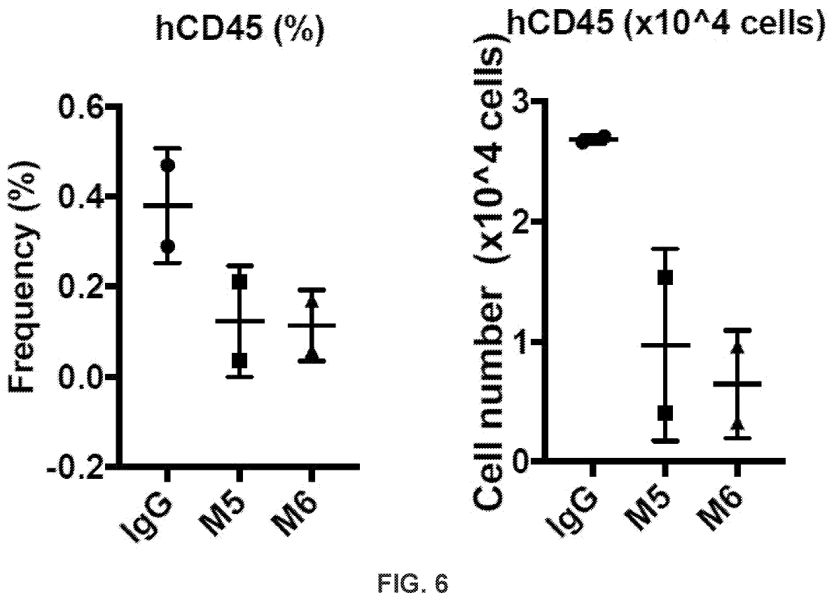

The impact of the anti-TSPAN3 mAbs against human leukemia samples was also tested in vivo. Initial analyses revealed responsiveness in some patient samples, an example of which is shown in FIG. 6. Briefly, mice were transplanted with primary patient AML; two days later either control IgG, M5 or M6 mAb, were administered (i.p., 15 mg/kg). This was followed by weekly dosing for three weeks (for a total of four mAb doses over the course of the experiment). Mice were sacrificed two days after the last mAb dose, and their bone marrow was analyzed by FACS. As shown, treatment of mice with the anti-TSPAN3 mAbs M5 or M6 impaired the growth of human AML in vivo.

Collectively, these data demonstrate the ability of the anti-TSPAN3 mAbs to block the growth of human myeloid leukemia cells (including both AML and bcCML) in vitro and in vivo. Because the monoclonal antibodies blocked the growth of human myeloid leukemia in vitro in colony assays and in vivo in patient-derived xenograft models, TSPAN3 represents a promising target for effective human cancer treatments, and therapeutics targeting TSPAN3 may be an effective new way to block the growth of aggressive cancers that express TSPAN3. When optimized and humanized for clinical use, anti-TSPAN3 antibodies have the potential to be used as anti-cancer therapeutics. Anti-TSPAN3 antibodies also have the potential to be used for antibody drug conjugates where a payload is targeted to cancer cells that express TSPAN3.

The above detailed description of embodiments of the technology is not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-10. (canceled)

11. An antibody that specifically binds to Tetraspanin-3 (TSPAN3), wherein the antibody comprises:
(a) a heavy chain comprising CDRs having amino acid sequences set forth in SEQ ID NOs: 3-5 and a light chain comprising CDRs having amino acid sequences set forth in SEQ ID NOs: 18-20;
(b) a heavy chain comprising CDRs having amino acid sequences set forth in SEQ ID NOs: 8-10 and a light chain comprising CDRs having amino acid sequences set forth in SEQ ID NOs: 28-30; or
(c) a heavy chain comprising CDRs having amino acid sequences set forth in SEQ ID NOs: 13-15 and a light chain comprising CDRs having amino acid sequences set forth in SEQ ID NOs: 33-35.

12. The antibody of claim 11, wherein the antibody comprises:
(a) a heavy chain variable region comprising an amino acid sequence that is at least 70% identical to an amino acid sequence set forth in SEQ ID NO: 2 and a light chain variable region comprising an amino acid sequence that is at least 70% identical to an amino acid sequence set forth in SEQ ID NO: 17;
(b) a heavy chain variable region comprising an amino acid sequence that is at least 70% identical to an amino acid sequence set forth in SEQ ID NO: 7 and a light chain variable region comprising an amino acid sequence that is at least 70% identical to an amino acid sequence set forth in SEQ ID NO: 27; or
(c) a heavy chain variable region comprising an amino acid sequence that is at least 70% identical to an amino acid sequence set forth in SEQ ID NO: 12 and a light chain variable region comprising an amino acid sequence that is at least 70% identical to an amino acid sequence set forth in SEQ ID NO: 32.

13. The antibody of claim 11, wherein the antibody is a monoclonal antibody.

14. The antibody of claim 11, wherein the antibody inhibits TSPAN3 function.

15. The antibody of claim 11, wherein the antibody is a humanized antibody.

16. A pharmaceutical composition comprising the antibody of claim 11.

17. (canceled)

18. A nucleic acid comprising a nucleotide sequence that encodes an antibody that specifically binds to Tetraspanin-3 (TSPAN3) or a fragment thereof, wherein the nucleotide sequence is at least 70% identical to a nucleotide sequence selected from the group consisting of SEQ ID NOs: 1, 6, 11, 16, 21, 26, and 31.

19. The nucleic acid of claim 18, wherein the nucleic acid is inserted into a vector.

20. A host cell containing the nucleic acid of claim 18.

21. (canceled)

22. A composition comprising the host cell of claim 20.

23. A method of treating and/or preventing a cancer that expresses TSPAN3 in a subject in need thereof, comprising administering to the subject a therapeutically effective amount of claim 11.

24. The method of claim 23, wherein the cancer is a hematologic malignancy.

25. The method of claim 24, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CIVIL), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

26. The method of claim 23, wherein the cancer is a solid cancer.

27. The method of claim 26, wherein the solid cancer is selected from the group consisting of pancreatic cancer, glioma, glioblastoma, colorectal cancer, thyroid cancer, stomach cancer, ovarian cancer, melanoma, endometrial cancer, lung cancer, renal cancer, cervical cancer, prostate cancer, breast cancer, urothelial cancer, testicular cancer, head and neck cancer, liver cancer, and esophageal cancer.


SEQUENCE LISTING (35 sequences)

SEQ ID NO: 1 - DNA, 360 bp, Artificial Sequence
  Synthetic Anti-TSPAN3 M1 clone heavy chain variable region DNA sequence
caggtccaactgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtcctaactacaatgagaggttcaagagcaaggccacactgactgtagacaaatcctccagcacagcctacatgcaactcagcagcctgacatctgaggactctgcggtctatttctgtgcaagatggggtgatggttaccacaggtacttcgatgtctggggcacagggaccacggtcaccgtctcctca

SEQ ID NO: 2 - protein, 120 aa, Artificial Sequence
  Synthetic Anti-TSPAN3 M1 clone heavy chain variable region amino acid sequence
QVQLQQPGAELVKPGASVKLSCKASGYTFTSYWMHWVKQRPGQGLEWIGMIHPNSGSPNYNERFKSKATLTVDKSSSTAYMQLSSLTSEDSAVYFCARWGDGYHRYFDVWGTGTTVTVSS

SEQ ID NO: 3 - protein, 8 aa, Artificial Sequence
  Synthetic Anti-TSPAN3 M1 clone heavy chain CDR1 amino acid sequence
GYTFTSYW

SEQ ID NO: 4 - protein, 8 aa, Artificial Sequence
  Synthetic Anti-TSPAN3 M1 clone heavy chain CDR2 amino acid sequence
IHPNSGSP

SEQ ID NO: 5 - protein, 13 aa, Artificial Sequence
  Synthetic Anti-TSPAN3 M1 clone heavy chain CDR3 amino acid sequence
ARWGDGYHRYFDV

SEQ ID NO: 6 - DNA, 350 bp, Artificial Sequence
  Synthetic Anti-TSPAN3 M3 clone heavy chain variable region DNA sequence
cagatccagttggtacagtctggacctgagctgaagaagcctggagagacagtcaagatctcctgcaaggcttctgggtataccttcacaacctatggaatgagctgggtgaaacaggctccaggaaagggtttaaagtggatgggctggataaacacctactctggagtgccaacatatgctgatgacttcaagggacggtttgccttctctttggaaacctctgccagcactgcctatttgcagatcaacaacctcaaaaatgaggacacggctacatatttctgtgcaagatcacattatagtaacttttatgctatggactactggggtcaagaacctcagtcacc

SEQ ID NO: 7 - protein, 117 aa, Artificial Sequence
  Synthetic Anti-TSPAN3 M3 clone heavy chain variable region amino acid sequence
QIQLVQSGPELKKPGETVKISCKASGYTFTTYGMSWVKQAPGKGLKWMGWINTYSGVPTYADDFKGRFAFSLETSASTAYLQINNLKNEDTATYFCARSHYSNFYAMDYWGQEPQSP

SEQ ID NO: 8 - protein, 8 aa, Artificial Sequence
  Synthetic Anti-TSPAN3 M3 clone heavy chain CDR1 amino acid sequence
GYTFTTYG

SEQ ID NO: 9 - protein, 8 aa, Artificial Sequence
  Synthetic Anti-TSPAN3 M3 clone heavy chain CDR2 amino acid sequence
INTYSGVP

SEQ ID NO: 10 - protein, 13 aa, Artificial Sequence
  Synthetic Anti-TSPAN3 M3 clone heavy chain CDR3 amino acid sequence
ARSHYSNFYAMDY

SEQ ID NO: 11 - DNA, 343 bp, Artificial Sequence
  Synthetic Anti-TSPAN3 M5/M6 clone heavy chain variable region DNA sequence
gaggtccagctgcaacagtctggacctgagctggtgaagcctggggcttcagtgaagatgtcctgcaaggcttctggatacacattcactgacttctacatgcattgggtgaagcagagtcatggaaggagccttgagtggattggatatatttaccctcacaatggttttactaagtacaaccagagtttcatgggcaaggccacattgactgtgaacaagtcctccaacacagcctacatggagctccgcagcctgacatcggaggattctgcagtctattactgtgcaagacgttacggggactactggggtcaaggaatctcagtcaccgtctcctcag

SEQ ID NO: 12 - protein, 114 aa, Artificial Sequence
  Synthetic Anti-TSPAN3 M5/M6 clone heavy chain variable region amino acid sequence
EVQLQQSGPELVKPGASVKMSCKASGYTFTDFYMHWVKQSHGRSLEWIGYIYPHNGFTKYNQSFMGKATLTVNKSSNTAYMELRSLTSEDSAVYYCARRYGDYWGQGISVTVSS

SEQ ID NO: 13 - protein, 8 aa, Artificial Sequence
  Synthetic Anti-TSPAN3 M5/M6 clone heavy chain CDR1 amino acid sequence
GYTFTDFY

SEQ ID NO: 14 - protein, 8 aa, Artificial Sequence
  Synthetic Anti-TSPAN3 M5/M6 clone heavy chain CDR2 amino acid sequence
IYPHNGFT

SEQ ID NO: 15 - protein, 7 aa, Artificial Sequence
  Synthetic Anti-TSPAN3 M5/M6 clone heavy chain CDR3 amino acid sequence
ARRYGDY

SEQ ID NO: 16 - DNA, 325 bp, Artificial Sequence
  Synthetic Anti-TSPAN3 M1 clone light chain variable region DNA sequence
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactcctccgtacacgttcggaggggggaccaagctggaaataaaac

SEQ ID NO: 17 - protein, 108 aa, Artificial Sequence
  Synthetic Anti-TSPAN3 M1 clone light chain variable region amino acid sequence
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPYTFGGGTKLEIK

SEQ ID NO: 18 - protein, 6 aa, Artificial Sequence
  Synthetic Anti-TSPAN3 M1 clone light chain CDR1 amino acid sequence
GNIHNY

SEQ ID NO: 19 - protein, 3 aa, Artificial Sequence
  Synthetic Anti-TSPAN3 M1 clone light chain CDR2 amino acid sequence
NAK

SEQ ID NO: 20 - protein, 10 aa, Artificial Sequence
  Synthetic Anti-TSPAN3 M1 clone light chain CDR3 amino acid sequence
QHFWSTPPYT

SEQ ID NO: 21 - DNA, 319 bp, Artificial Sequence
  Synthetic Anti-TSPAN3 M2 clone light chain variable region DNA sequence
gaaattgtgctcactcagtctccagccatcacagctgcatctctggggcaaaaggtcaccatcacctgcagtgccagctcaagtgtaagttacatgaactggtaccagcagaagtcaggcacctcccccaaaccatggatttatgaaatatccaaactggcttctggagtcccagttcgcttcagtggcagtgggtctgggacctcttactctctcacaatcagcagcatggaggctgaagatgctgccatttattactgccagcagtggaattatcctcttatcacgttcggaggggggaccaagctggaaataaaac

SEQ ID NO: 22 - protein, 106 aa, Artificial Sequence
  Synthetic Anti-TSPAN3 M2 clone light chain variable region amino acid sequence
EIVLTQSPAITAASLGQKVTITCSASSSVSYMNWYQQKSGTSPKPWIYEISKLASGVPVRFSGSGSGTSYSLTISSMEAEDAAIYYCQQWNYPLITFGGGTKLEIK

SEQ ID NO: 23 - protein, 5 aa, Artificial Sequence
  Synthetic Anti-TSPAN3 M2 clone light chain CDR1 amino acid sequence
SSVSY

SEQ ID NO: 24 - protein, 3 aa, Artificial Sequence
  Synthetic Anti-TSPAN3 M2 clone light chain CDR2 amino acid sequence
EIS

SEQ ID NO: 25 - protein, 9 aa, Artificial Sequence
  Synthetic Anti-TSPAN3 M2 clone light chain CDR3 amino acid sequence
QQWNYPLIT

SEQ ID NO: 26 - DNA, 322 bp, Artificial Sequence
  Synthetic Anti-TSPAN3 M3 clone light chain variable region DNA sequence
gacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatccgctcacgttcggtgctgggaccaagctggagctgaaac

SEQ ID NO: 27 - protein, 107 aa, Artificial Sequence
  Synthetic Anti-TSPAN3 M3 clone light chain variable region amino acid sequence
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELK

SEQ ID NO: 28 - protein, 6 aa, Artificial Sequence
  Synthetic Anti-TSPAN3 M3 clone light chain CDR1 amino acid sequence
QDVGTA

SEQ ID NO: 29 - protein, 3 aa, Artificial Sequence
  Synthetic Anti-TSPAN3 M3 clone light chain CDR2 amino acid sequence
WAS

SEQ ID NO: 30 - protein, 9 aa, Artificial Sequence
  Synthetic Anti-TSPAN3 M3 clone light chain CDR3 amino acid sequence
QQYSSYPLT

SEQ ID NO: 31 - DNA, 334 bp, Artificial Sequence
  Synthetic Anti-TSPAN3 M5/M6 clone light chain variable region DNA sequence
gacattgtgctgacccaatctccagcttctttggctgtgtctctggggcagagggccaccatctcctgcagagccagcgaaagtcttggtaattatggcattagctatatgaactggttccaacagaaaccaggacagccacccaaactcctcatctatgctgcatccaaccaaggatccggggtccctgtcaggtttagtggcagtgggtccgggacagacttcagcctcaacatccatcctatggaggaggatgatactgcaatgtatttctgtcagcaaagtaaggaagttccgtggacgttcggtggaggcaccaagctggaaatcaaac

SEQ ID NO: 32 - protein, 111 aa, Artificial Sequence
  Synthetic Anti-TSPAN3 M5/M6 clone light chain variable region amino acid sequence
DIVLTQSPASLAVSLGQRATISCRASESLGNYGISYMNWFQQKPGQPPKLLIYAASNQGSGVPVRFSGSGSGTDFSLNIHPMEEDDTAMYFCQQSKEVPWTFGGGTKLEIK

SEQ ID NO: 33 - protein, 10 aa, Artificial Sequence
  Synthetic Anti-TSPAN3 M5/M6 clone light chain CDR1 amino acid sequence
ESLGNYGISY

SEQ ID NO: 34 - protein, 3 aa, Artificial Sequence
  Synthetic Anti-TSPAN3 M5/M6 clone light chain CDR2 amino acid sequence
AAS

SEQ ID NO: 35 - protein, 9 aa, Artificial Sequence
  Synthetic Anti-TSPAN3 M5/M6 clone light chain CDR3 amino acid sequence
QQSKEVPWT